(19) United States
(12) Patent Application Publication
(10) Pub. No.: US 2017/0372472 A1
(43) Pub. Date: Dec. 28, 2017

(54) Medical Image Processing Apparatus And Medical Image Capturing System

(71) Applicant: KONICA MINOLTA, INC., Chiyoda-ku (JP)

(72) Inventors: Atsushi TAKAHASHI, Musashino-shi (JP); Yoshihide HOSHINO, Chiyoda-ku (JP)

(73) Assignee: KONICA MINOLTA, INC., Chiyoda-ku (JP)

(21) Appl. No.: 15/623,067

(22) Filed: Jun. 14, 2017

(30) Foreign Application Priority Data
Jun. 23, 2016 (JP) 2016-124036

(51) Int. Cl. G06T 7/00; A61B 6/00

(52) U.S. Cl. CPC G06T 7/0012; A61B 6/505; A61B 6/5217; A61B 6/4035; A61B 6/484; G06T 2207/30008

(57) ABSTRACT

A medical image processing apparatus includes a retriever, a determiner and a calculator. The retriever retrieves a medical image of a bone and a soft tissue of a subject. The determiner determines a position of at least one of an end of the bone and an end of the soft tissue based on the medical image retrieved by the retriever. The calculator calculates a feature value relevant to a condition of the soft tissue based on the position of at least one of the end of the bone and the end of the soft tissue determined by the determiner.

### BACKGROUND OF THE INVENTION

### 1. Field of the Invention

The present invention relates to a medical image processing apparatus and a medical image capturing system.

### 2. Description of the Related Art

Simple X-ray images (i.e., absorption images) usually captured on traditional silver halide film fails to visualize joint cartilage in patients. Magnetic resonance imaging (MRI) can visualize joint cartilage. Unfortunately, the moderate resolution of the MRI often prevents quantitative measurements of defects, damage, and wear of the cartilage due to arthritis or arthrorheumatism in a relatively small joint, such as a finger joint. Thus, in traditional diagnosis of arthrorheumatism in a finger, the relevant finger of the patient is irradiated with X-rays to capture a simple X-ray image, and the thickness and the condition, such as wear, of the cartilage are estimated from the gap between the bones in the joint of the finger without visualization of the cartilage in the image.

U.S. Pat. No. 5,812,629, Japanese Patent Application Laid-Open Publication No. 2008-200359, for example, and International Publication No. WO2011/033798 disclose X-ray image capturing apparatuses that include Talbot interferometers based on the Talbot effect and capture phase shifts in X-rays passing through subjects, and X-ray image capturing apparatuses including Talbot-Lau interferometers. The X-ray image capturing apparatus including a Talbot interferometer or a Talbot-Lau interferometer is hereinafter referred to as an X-ray Talbot image capturing apparatus.

An X-ray Talbot image capturing apparatus captures moire images. One or more moire images captured through a method based on the principle of fringe scanning are analyzed through Fourier transform to reconstruct the moire images into at least three different types of images: an absorption image (which is the same as the absorption image mentioned above) that visualize a contrast based on the absorption of X-rays; a differential phase image that visualizes a contrast based on phase information; and a small-angle scattering image that visualizes a contrast based on small-angle scattering.

The inventors of the present invention applied the X-ray Talbot image capturing apparatus to image capturing of joint cartilage and captured an image of a dissected joint with the X-ray Talbot image capturing apparatus and discovered that the joint cartilage can be visualized in at least differential phase images, as described in Nagashima M. et al (2010). Optimization of the joint and cartilage: diagnostic potential of the differential interferential contrast X-ray imaging. 14th Japanese Research Society of Clinical Anatomy Meeting Kanazawa, Sep. 11, 2010 Abstract Book (February 2011), No. 11, 56-57. Retrieved from http://www.jrsca.jp/contents/records/on Nov. 21, 2013. Besides the dissected joint described above, the inventors also have captured moire images of a joint of an undissected living body and reconstructed images as described above. As a result, the inventors have discovered that joint cartilage can be visualized in at least differential phase images.

Japanese Patent Application Laid-Open Publication No. 2015-104441, for example, proposes a medical image capturing system that measures the thickness of joint cartilage in reconstructed images, such as absorption images and differential phase images.

The medical image capturing system disclosed in Japanese Patent Application Laid-Open Publication No. 2015-104441 can measure the distance between the end of the joint bone to the end of the cartilage captured in a reconstructed image but fails to measure the quantitative condition of the entire cartilage. A patient suffering from a disease may have a depression in the surface of a joint cartilage, for example. A reconstructed image can visualize the depression at the end of the cartilage but fails to three-dimensionally visualize the cartilage. Thus, an expansion of the depression in the anteroposterior direction of the cartilage cannot be confirmed in the reconstructed image. The pixel values corresponding to the end of the cartilage may be relatively small depending on the surface condition of the cartilage. This prevents positioning of the end of the cartilage.

### SUMMARY OF THE INVENTION

### Problem to be Solved by the Invention

An object of the present invention, which has been accomplished to solve the drawbacks described above, is to provide a medical image processing apparatus and a medical image capturing system that can quantitatively measure the condition of soft tissue captured in a medical image.

### Means for Solving the Problem

To solve the above object, according to one aspect of a preferred embodiment of the present invention, there is provided a medical image processing apparatus including: a retriever that retrieves a medical image of a bone and a soft tissue of a subject; a determiner that determines a position of at least one of an end of the bone and an end of the soft tissue based on the medical image retrieved by the retriever; and a calculator that calculates a feature value relevant to a condition of the soft tissue based on the position of at least one of the end of the bone and the end of the soft tissue determined by the determiner.

### BRIEF DESCRIPTION OF THE DRAWINGS

The present invention will be described through the detailed description below and the accompanying drawings. Such description and drawings should not be construed to limit the present invention, wherein

FIG. 1 is a schematic view of the overall configuration of a medical image capturing system according to an embodiment;

FIG. 2 is a block diagram illustrating the functional configuration of a medical image processing apparatus;

FIG. 3A illustrates an example absorption image;

FIG. 3B illustrates an example differential phase image;

FIG. 4 illustrates an example polar coordinate converted image obtained through polar conversion of the differential phase image;

FIG. 5A illustrates a portion of an enlarged polar coordinate converted image;

FIG. 5B illustrates pixel values corresponding to a portion of the polar coordinate converted image;

FIG. 6 illustrates pixel values of a portion of an enlarged polar coordinate converted image;

FIG. 7A illustrates representative pixel values of the rows in one of the columns in the polar coordinate converted image;

FIG. 7B illustrates the pixel values of the rows in one of the columns in the polar coordinate converted image;

FIG. 8A illustrates the position of the end of cartilage in a differential phase image;

FIG. 8B illustrates the position of the end of the cartilage in a polar coordinate converted image;

FIG. 8C illustrates an example standard deviation of the thicknesses of the cartilage;

FIG. 9 illustrates the pixel values corresponding to a portion of a polar coordinate converted image acquired through polar conversion of a differential phase image;

FIG. 10A illustrates representative pixel values in a range having a first width and another range having a second width in one of the columns in the polar coordinate converted image;

FIG. 10B illustrates example differences between the representative pixel values in the ranges having the first and second widths;

FIG. 11 illustrates an example standard deviation of the differences between the representative pixel values of the ranges having the first and second widths in multiple columns of the polar coordinate converted image;

FIGS. 12A and 12B illustrate example pixel values of the rows in one of the columns of the polar coordinate converted image; and

FIG. 13 illustrates example conditions before and after interpolation of pixel values of the rows of one of the columns of the polar coordinate converted image.

### DETAILED DESCRIPTION OF THE PREFERRED EMBODIMENTS

A medical image capturing system according to embodiments of the present invention will now be described with reference to the accompanying drawings.

[Configuration of Medical Image Capturing System]

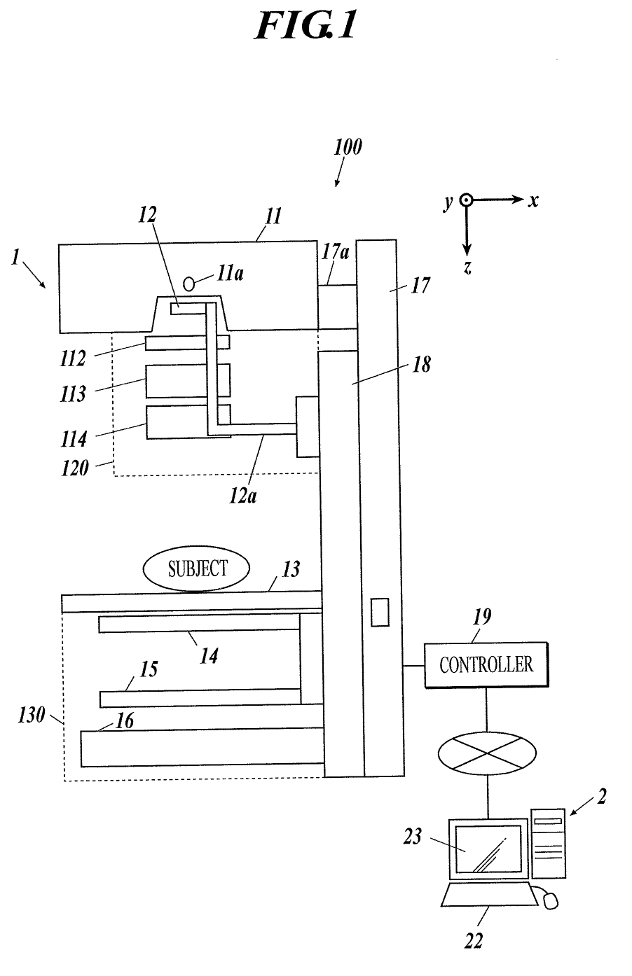

FIG. 1 is a schematic view of the overall configuration of a medical image capturing system 100 according to an embodiment.

With reference to FIG. 1, the medical image capturing system 100 according to this embodiment includes an X-ray Talbot image capturing apparatus 1 and a medical image processing apparatus 2.

[X-ray Talbot Image Capturing Apparatus]

The X-ray Talbot image capturing apparatus 1 according to this embodiment includes a Talbot-Lau interferometer including a source grating (also referred to as multigrating or multislit) 12, as described below. Alternatively, the X-ray Talbot image capturing apparatus 1 may include a Talbot interferometer including only a first grating (G1 grating) 14 and a second grating (G2 grating) 15 without the source grating 12.

The Talbot effect, which is the basis of the configuration of the Talbot interferometer, is a phenomenon in which coherent X-ray beams passing through a first grating (G1 grating) having slits disposed at a predetermined pitch form images of the grating along the traveling direction of the X-ray beams at a constant period (refer to Japanese Patent Application Laid-Open Publication No. 2008-200359, for example). The grating images are referred to as self-images. In a Talbot interferometer, a second grating (G2 grating) is placed at the position of the self-image to form moire stripes. A subject placed at a position within the range of the irradiated X-ray beams causes distortion in the moire stripes.

The X-ray Talbot image capturing apparatus 1 captures moire images of moire stripes distorted by the subject.

The configuration of the X-ray Talbot image capturing apparatus 1 will now be described.

The X-ray Talbot image capturing apparatus 1 includes a radiation generator 11, a source grating 12, a subject bed 13, a first grating 14, a second grating 15, a radiation detector 16, a supporting column 17, a base 18, and a controller 19.

The radiation generator 11 disposed at the top of the X-ray Talbot image capturing apparatus 1 according to this embodiment emits radiation downward toward the subject disposed below the radiation generator 11, as illustrated in FIG. 1. Alternatively, the radiation generator 11 of the X-ray Talbot image capturing apparatus 1 according to the present invention may emit radiation in the horizontal direction or any other direction.

The radiation generator 11 includes an X-ray source 11a that is a Coolidge X-ray source or a rotary anode X-ray source, for example, which are commonly used in medical settings. Alternatively, any other X-ray source may be used. With reference to FIG. 1, the source grating 12 is disposed below the radiation generator 11.

The source grating 12 is fixed to a fixing member 12a extending from the base 18 fixed to the supporting column 17. The source grating 12 is not fixed to the radiation generator 11 so as to prevent transmission of vibration of the radiation generator 11 caused by the rotation of the anode of the X-ray source 11a to the source grating 12. A buffer 17a is disposed between the radiation generator 11 and the supporting column 17 to eliminate or damp vibration of the radiation generator 11 transmitted to the other components of the X-ray Talbot image capturing apparatus 1, such as the supporting column 17.

The source grating 12, the first grating 14, and the second grating 15 each have multiple slits (not shown) at a predetermined pitch in the y direction, which is orthogonal to the z direction or direction of radiation. The slits extend in the x direction.

The fixing member 12a is provided with a filter (also referred to as additional filter) 112 that modifies the quality of radiation passing through the source grating 12, a collimator 113 that narrows the radiation field, and a radiation field lamp 114 that irradiates the subject with visible light for alignment before radiation irradiation, in addition to the source grating 12. The source grating 12, the filter 112, and the collimator 113 may be disposed in this order or any other order.

The source grating 12 and other components are covered with a first cover unit 120 for protection.

The subject bed 13 is disposed between the radiation generator 11 and the first grating 14. The subject or patient is disposed on the subject bed 13 for image capturing of a joint of the patient. With reference to FIG. 1, the first grating 14 and the second grating 15 are disposed below the subject bed 13, and the radiation detector 16 is disposed directly below the second grating 15.

The radiation detector 16 includes transducers (not shown) that generate electric signals in proportion to the incident X-rays and reads the electrical signals generated at the transducers as image signals. The radiation detector 16 captures the moire images formed on the second grating 15. The first grating 14, the second grating 15, and the radiation detector 16 are covered with a second cover unit 130 that protect them from the legs of the patient or subject.

To capture multiple moire images through fringe scanning, the X-ray Talbot image capturing apparatus 1 should include a shifter (not shown) that shifts one of the source grating 12, the first grating 14, and the second grating 15 or both of the first grating 14 and the second grating 15 in the y direction. Alternatively, the X-ray Talbot image capturing apparatus 1 according to the present invention may capture a single moire image without fringe scanning, and the controller 19 may analyze the moire image through Fourier transform to reconstruct an absorption image or a differential phase image.

The controller 19 includes a computer (not shown) provided with a central processing unit (CPU), a read only memory (ROM), a random access memory (RAM), and an input/output interface connected to a bus. Alternatively, the controller 19 may be a dedicated control device. Although not illustrated, the controller 19 includes appropriate units and devices, such as an input unit and a display unit. The controller 19 comprehensively controls the X-ray Talbot image capturing apparatus 1.

In specific, the controller 19 establishes the tube voltage and irradiation time of the radiation generator 11, for example. The controller 19 also controls the distance and rate of the shift of the first grating 14 by the shifter and adjusts the timing of the shift of the grating and the irradiation of radiation from the radiation generator 11, if the X-ray Talbot image capturing apparatus 1 captures multiple moire images through fringe scanning, as described above.

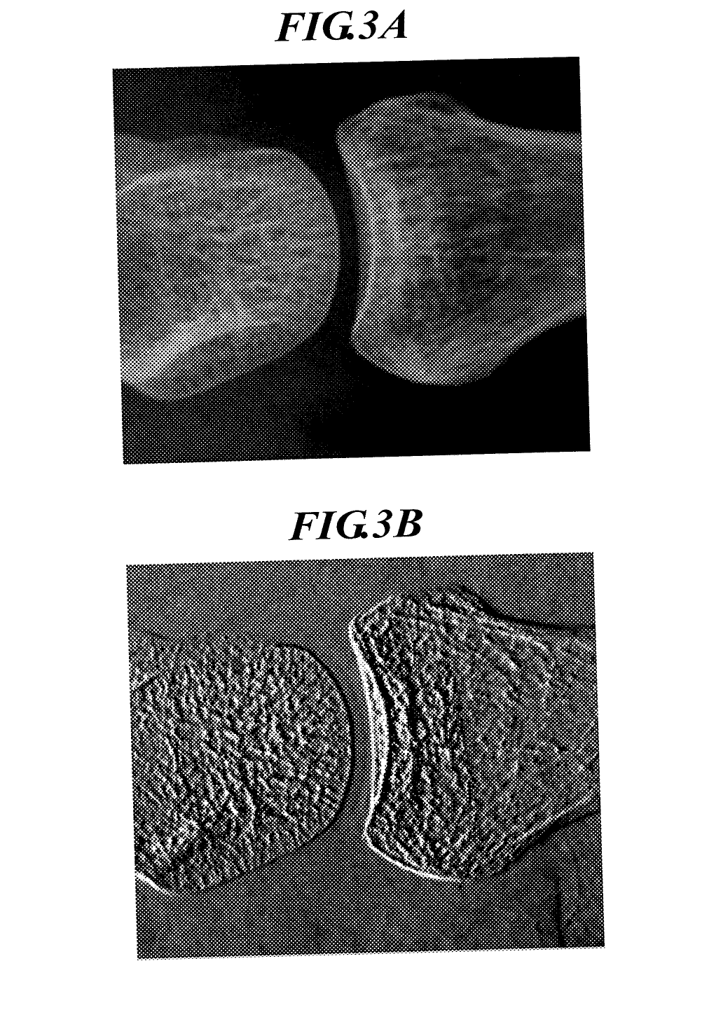

The controller 19 reconstructs an X-ray absorption image (see FIG. 3A), a differential phase image (see FIG. 3B), and a small-angle scattering image (not shown), on the basis of a single moire image or multiple moire images. The controller 19 reconstructs an absorption image, a differential phase image, and a small-angle scattering image (and various images generated through combination of these images) of a subject, on the basis of the image signals corresponding to the subject captured by the radiation detector 16, i.e., moire images.

The absorption image in FIG. 3A and the differential phase image in FIG. 3B are example images reconstructed on the basis of moire images of a finger joint of a subject or patient.

The controller 19 is connected to the medical image processing apparatus 2 (which is described in detail below) via a network, such as a local area network (LAN).

In this embodiment, the controller 19 and the medical image processing apparatus 2 are separate components. Alternatively, the controller 19 and the medical image processing apparatus 2 may be integrated into a single component. A generator (not shown) that controls the radiation generator 11 may be provided separately from the controller 19. In other words, two or more of the controller 19, the medical image processing apparatus 2, and the generator of the radiation generator 11 may be integrated into a single component, or the controller 19, the medical image processing apparatus 2, and the generator of the radiation generator 11 may be provided in the form of independent components.

[Medical Image Processing Apparatus]

The configuration of the medical image processing apparatus 2 will now be described.

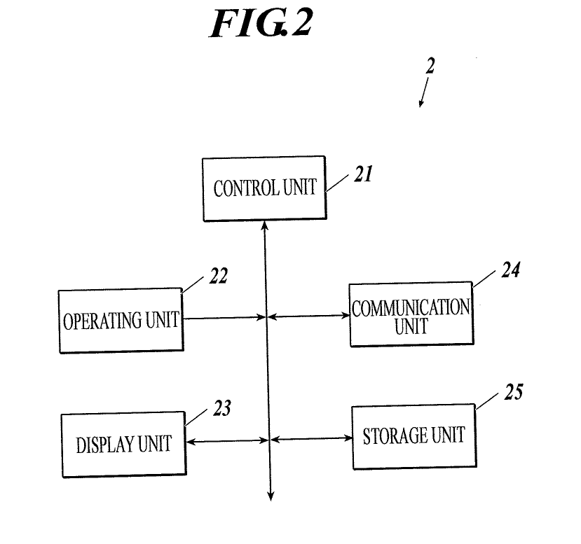

FIG. 2 is a block diagram illustrating the functional configuration of the medical image processing apparatus 2.

The medical image processing apparatus 2 includes a general-purpose computer and a dedicated processor, as similar to the controller 19 of the X-ray Talbot image capturing apparatus 1. In detail, the medical image processing apparatus 2 includes a control unit 21, an operating unit 22, a display unit 23, a communication unit 24, and a storage unit 25, as illustrated in FIG. 2.

The control unit 21 includes a central processing unit (CPU) and a random access memory (RAM). The control unit 21 operates in cooperation with programs stored in the storage unit 25 to execute various processes. The control unit 21 executes process of measuring soft tissue, which is described in detail below, to calculate the feature value relevant to the condition of the soft tissue in the joint, on the basis of reconstructed images (medical images) of the joint of a subject or patient, such as an absorption image (see FIG. 3A), a differential phase image (see FIG. 3B), and a small-angle scattering image.

The operating unit 22 includes a keyboard and a mouse, for example. The operating unit 22 generates operational signals corresponding to certain operations and outputs the operational signals to the control unit 21.

The display unit 23 causes various images and graphs to appear on a display, such as a cathode ray tube (CRT) or liquid crystal display (LCD), in accordance with the display control by the control unit 21.

The communication unit 24 includes a communication interface and communicates with external units in the network (such as the controller 19 of the X-ray Talbot image capturing apparatus 1).

The storage unit 25 stores programs to be executed by the control unit 21 and data required for the execution of the programs.

[Process of Measuring Soft Tissue]

The soft-tissue measuring process executed by the control unit 21 of the medical image processing apparatus 2 will now be described in detail.

In specific, the controller 19 of the X-ray Talbot image capturing apparatus 1 generates reconstructed images (medical images), such as an absorption image (see FIG. 3A), a differential phase image (see FIG. 3B), and a small-angle scattering image, on the basis of moire images of the finger joint of a subject or patient, for example, as described above. The differential phase image is known to visualize an end corresponding to the surface of the joint cartilage (hereinafter referred to as the end of the cartilage), for example. The inventors have developed a technique for measuring the thickness of the cartilage through measurement of the distance between the end of the bone and the end of the cartilage. In actual medical settings, there is also a need for quantitative measurements of the condition of the entire soft tissue, including the thickness of the cartilage.

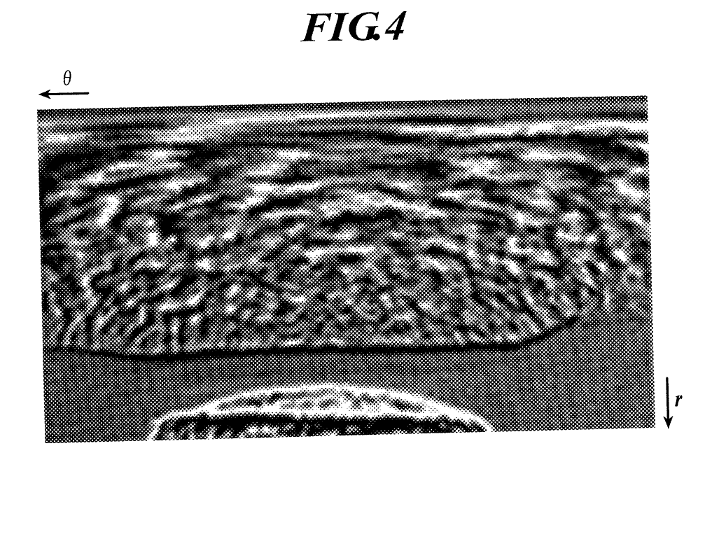

In this embodiment, the control unit 21 of the medical image processing apparatus 2 calculates the feature values relevant to the condition of the soft tissue by reference to the pixel values of a polar coordinate converted image (see FIG. 4) acquired through polar conversion of a reconstructed image, such as an absorption image (see FIG. 3A), a differential phase image (see FIG. 3B), or a small-angle scattering image (process of measuring soft tissue). In the process of measuring soft tissue, the control unit 21 functions as a retriever, a determiner, and a calculator.

The function and the operation of the control unit 21 in the process of measuring soft tissue will now be described in first, second, and third schemes.

The reconstructed image is exemplified by a differential phase image (see FIG. 3B) in the description below. Alternatively, the reconstructed image may be an absorption image (see FIG. 3A), a small-angle scattering image, or an image reconstructed from the absorption image, the differential phase image, or any other image. An absorption image may be used only for detection of the end of the bone because it fails to clearly visualize soft tissue.

<First Scheme>

In the first scheme, the control unit 21 calculates the feature value relevant to the condition of the soft tissue along the direction of an angle θ around the center of the arc in the polar coordinate converted image. In detail, the control unit 21 calculates the thicknesses of the cartilage in multiple columns corresponding to multiple angles θ around the center of the arc in the polar coordinate converted image and thereby calculates the degree of variance in the calculated thicknesses of the cartilage in the multiple columns, as a feature value (first feature value) relevant to the condition of the soft tissue along the direction of the angles θ around the center of the arc.

The first feature value is a feature value relevant to the condition of the soft tissue extending along the contour of the end of the bone. The first feature value will be described in detail below.

In specific, the control unit (retriever) 21 retrieves the differential phase image (see FIG. 3B) reconstructed by the controller 19 of the X-ray Talbot image capturing apparatus 1 via the communication unit 24. The control unit 21 may instruct the display unit 23 to display the retrieved differential phase image on the display.

The control unit (determiner) 21 then determines the position of the end of the bone on the basis of the retrieved differential phase image.

In specific, among the pixel values of the pixels detected one by one in order from the left to right along a pixel row having a width of one pixel in the differential phase image, the pixel corresponding to the end of the joint bone has a pixel value significantly smaller than that of the preceding pixel. Thus, a threshold may be established for the variation in the pixel values. The pixel values in the differential phase image are detected one by one in order from, for example, the left to the right of the pixel row having a width of one pixel to calculate the average pixel value of the pixels. Alternatively, the moving average of 30 or 100 pixels, for example, may be calculated instead of the average of pixel values. If the absolute value of the difference between a pixel value of a pixel and the calculated average or moving average (excluding the pixel) is larger than or equal to the threshold, the pixel having a pixel value significantly larger than or equal to the threshold is determined to be the position of the end of the joint bone in the pixel row. This process can be continued with the other pixel rows in the differential phase image by vertically shifting to the pixel row to be processed, to determine the position of the end of the joint bone in the differential phase image.

The control unit 21 may instruct the display unit 23 to display the differential phase image on the display such that the determined end of the joint bone is identifiable in the image.

Alternatively, the reconstructed image may be an absorption image (see FIG. 3A). In such a case, the pixel corresponding to the end of the joint bone has a pixel value significantly smaller (are darker) than that of the preceding pixel, among the pixel values of the pixels detected one by one in order from the left to right, for example, along the pixel row having a width of one pixel in the absorption image.

In the absorption image (see FIG. 3A) of the finger with the left and right reversed in the image, the pixels corresponding to the bone have large pixel values (are bright), and the pixels surrounding the pixels corresponding to the bone have small pixel values. In contrast, in a differential phase image (see FIG. 3B) of the finger with the left and right reversed in the image, the brightness of the pixels at the end of the joint bone and the vicinity is reversed. With reference to the differential phase image in FIG. 3B, the pixels corresponding to the end of the joint bone on the left of the image have small pixel values (are dark), whereas the pixels corresponding to the end of the joint bone on the right have large pixel values (are bright).

Thus, if the bone having a protruding articular surface (i.e., the bone having the articular head) is captured on the left of the differential phase image (see FIG. 3B), the pixel having a pixel value significantly smaller than that of the preceding pixel among the pixels detected one by one in order from the left to right along a pixel row having a width of one pixel in the differential phase image is determined to correspond to the end of the joint bone. On the contrary, although not illustrated, if the bone having the protruding articular surface is captured on the right of the differential phase image, the pixel having a pixel value significantly larger than that of the preceding pixel among the pixels detected one by one in order from the right to left along a pixel row having a width of one pixel in the differential phase image is determined to correspond to the end of the joint bone. In the cases described above, the joint is disposed horizontally. If the joint is disposed vertically, the same procedures still apply but in a different direction.

As described above, the average or moving average of the pixel values of the pixels in a predetermined pixel row of the differential phase image and the difference (or the absolute value of the difference) between the average or moving average and each pixel value are calculated because every reconstructed image, such as differential phase images, have different overall brightness.

In place of a threshold for the difference, a threshold for the quotient of the difference and the average or the moving average (which is the rate of variation) may be determined. The pixel having a rate of variation (or an absolute value of the rate of variation) larger than or equal to the threshold can be determined to correspond to the position of the end of the joint bone. Alternatively, the end of the joint bone may be determined by the image processing apparatus 2 through any other scheme.

Alternative to the automatic determination of the end of the joint bone by the image processing apparatus 2 as described above, manual determination may be carried out by a user viewing the reconstructed image, such as the differential phase image, displayed on the display unit 23 of the image processing apparatus 2, for example.

In the determination process of the end of the joint bone, not only the end of the bone having a protruding articular surface (i.e., the bone on the left in FIGS. 3A and 3B) but also the bone having a depressed articular surface (i.e., the bone having an articular fossa such as that on the right in FIGS. 3A and 3B) may be detected. Thus, if the ends of multiple bones are to be determined in the reconstructed image, such as the differential phase image, the ends of the bones are determined to be either protruding or depressed, and the bone having a protruding end may be determined to be the end of the joint bone.

The reconstructed images, such as the absorption image, the differential phase image, and the small-angle scattering image, are all reconstructed from the same moire image captured by the X-ray Talbot image capturing apparatus 1. Thus, the end of the joint bone resides at the same position in every reconstructed image. Thus, information on the position of the end of the joint bone (i.e., the coordinates of the pixels in the reconstructed image, for example) determined as described above can be directly applied to the differential phase image, to determine the position of the end of the joint bone in the differential phase image.

The control unit (polar coordinate converter) 21 then approximates the end of the joint bone determined in the differential phase image to an arc and converts the coordinates of the arc to polar coordinates including the angle θ around the center of the arc and the distance r from the center of the arc.

In detail, the control unit 21 automatically selects at least three points on the end of the joint bone in the differential phase image or selects at least three points in response to a user operation of the operating unit 22. The control unit 21 then approximates the end of the joint bone to an arc on the basis of the selected at least three points and converts the coordinates of the arc to polar coordinates (θ, r), where θ is a component of the angle around the center of the arc and r is a component of the distance from the center of the arc (see FIG. 4). FIG. 4 illustrates an example polar coordinate converted image acquired through polar conversion of a differential phase image, where the horizontal direction represents the direction of the θ axis corresponding to the angle θ around the center of the arc, and the vertical direction represents the direction of the r axis corresponding to the distance r from the center of the arc.

The end of the bone having a protruding articular surface in Cartesian coordinates (exactly the portion that can be satisfactorily being approximation of the arc (see FIG. 3B)) is represented in the form of a substantially straight line in the polar coordinate converted image. Similarly, for example, the end of the cartilage of a normal subject without a disease, such as arthrorheumatism, is represented in the form of a substantially straight line parallel to the substantially straight line representing the end of the bone.

Alternatively, the control unit 21 may select multiple points on the end of the joint bone in the differential phase image for approximation of the end of the joint bone to an arc through the method of least squares or Hough transform, for example.

The control unit (determiner) 21 determines the positions of the end of the joint bone and the end of the cartilage in each column corresponding to each angle θ around the center of the arc in the polar coordinate converted image, on the basis of the pixel values in the polar coordinate converted medical image (polar coordinate converted image) (see FIG. 5B).

The control unit 21 needs not to use the pixel values of the entire polar coordinate converted image for the direction of the r-axis. This is because the center of the arc is usually located deep in the bone away from the end, and thus the area between the center of the arc and the end of the bone is not directly involved with the measurement of the condition of soft tissue, which is an object of the present invention. Thus, the control unit 21 may refer to the positional information (coordinates) of the pixels corresponding to the end of the joint bone determined in the differential phase image, for example, and determine the position of the corresponding pixels in the polar coordinate converted image as the position of the end of the bone in the polar coordinate converted image illustrated in FIG. 5A, and retrieve pixel values of the pixels within a predetermined range at least from near the end of the bone toward the cartilage (in the direction away from the center of the arc or toward the bottom of FIG. 4).

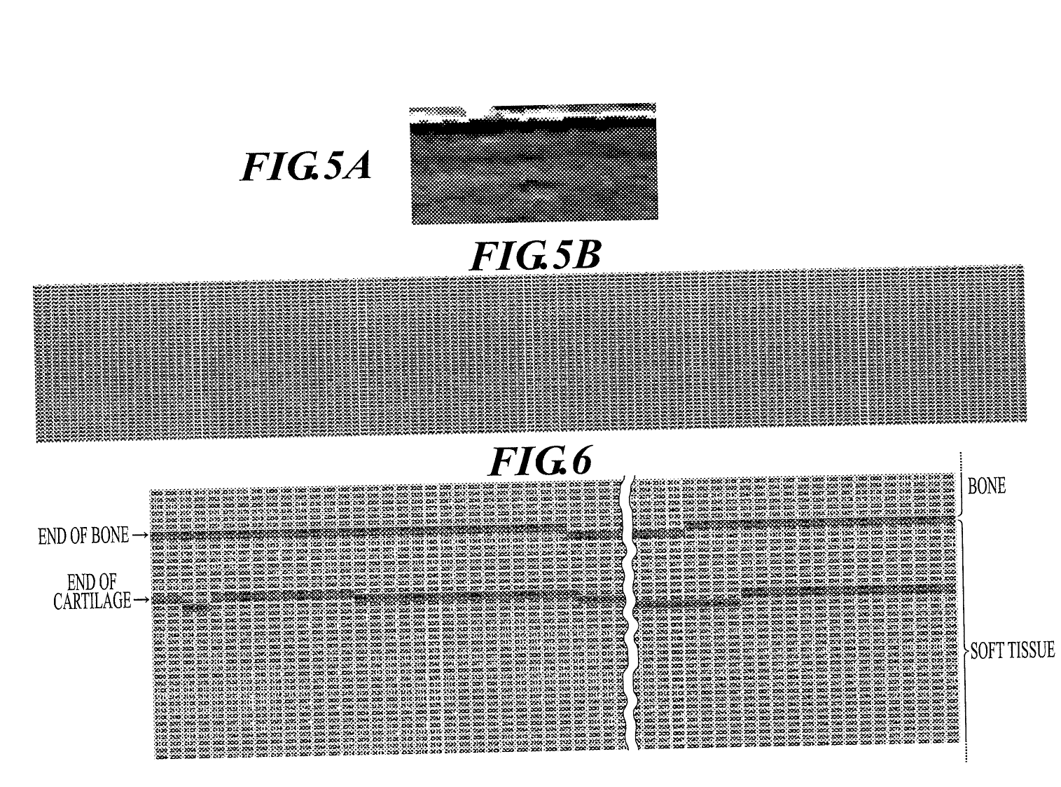

FIG. 5B illustrates the portion of the polar coordinate converted image illustrated in FIG. 5A converted to pixel values in accordance with a predetermined gradation (such as a 16-bit gradation). FIG. 6 illustrates the pixel values of a portion of the polar coordinate converted image in FIG. 5B in an enlarged state, as described below.

In detail, the pixel values are small at the end of the joint bone in the differential phase image in which the bone having a protruding articular surface (i.e., the bone having the articular head) resides on the left, as described above. Similarly, the pixel values are small at the end of the joint bone in the polar coordinate converted image, which is acquired through polar conversion of the differential phase image. Thus, the control unit 21 detects the pixel values of the pixels one by one in order in the direction away from the center of the arc (from the top to bottom in the drawing) in each row corresponding to a distance r from the center of the arc in each column corresponding to an angle θ around the center of the arc in the polar coordinate converted image and determines the pixel in the row having the minimum value at the position closest to the center of the arc, as the position of the end of the bone (see FIG. 6). Although the minimum value at a position closest to the center of the arc may not necessarily correspond to the end of the bone, the area near the end of the bone has a mesh-like structure and is highly likely to have a minimum pixel value.

The control unit 21 determines the pixel in the row having the second smallest value to the value corresponding to the end of the bone in each column corresponding to each angle θ around the center of the arc in the polar coordinate converted image, as the position of the end of the cartilage (see FIG. 6).

Although not illustrated, the pixel values are large at the end of the joint bone in the differential phase image in which the bone having a protruding articular surface resides on the right. Similarly, the pixel values are large at the end of the joint bone in the polar coordinate converted image, which is acquired through polar conversion of the differential phase image. In such the case, the control unit 21 determines the pixel in the row having the maximum value at the position closest to the center of the arc in each column corresponding to each angle θ around the center of the arc in the polar coordinate converted image, as the position of the end of the bone. The control unit 21 determines the pixel in the row having the second largest value to the value corresponding to the end of the bone in each column corresponding to each angle θ around the center of the arc in the polar coordinate converted image, as the position of the end of the cartilage.

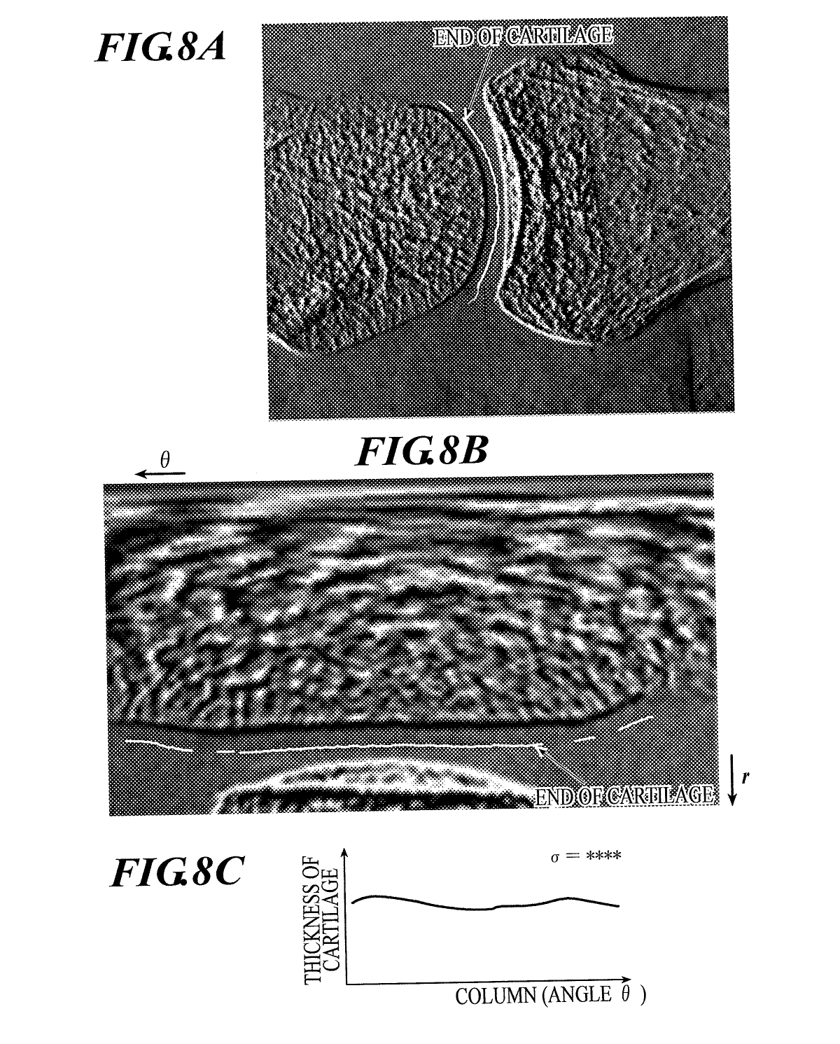

The control unit 21 may instruct the display unit (output unit) 23 to display the determined position of the end of the joint cartilage (indicated by the arrows in the drawings) in the differential phase image or the polar coordinate converted image on the display such that the position of the end of the joint cartilage is identifiable (see FIGS. 8A and 8B). With reference to FIGS. 8A and 8B, the end of the cartilage is indicated by white lines. Alternatively, the end of the cartilage may be indicated in any way identifiable by a user.

The position of the end of the bone in the polar coordinate converted image may be determined by reference to the positional information (coordinates) of the pixels representing the end of the joint bone in the differential phase image and determining the position of the corresponding pixels in the polar coordinate converted image as the position of the end of the bone, for example.

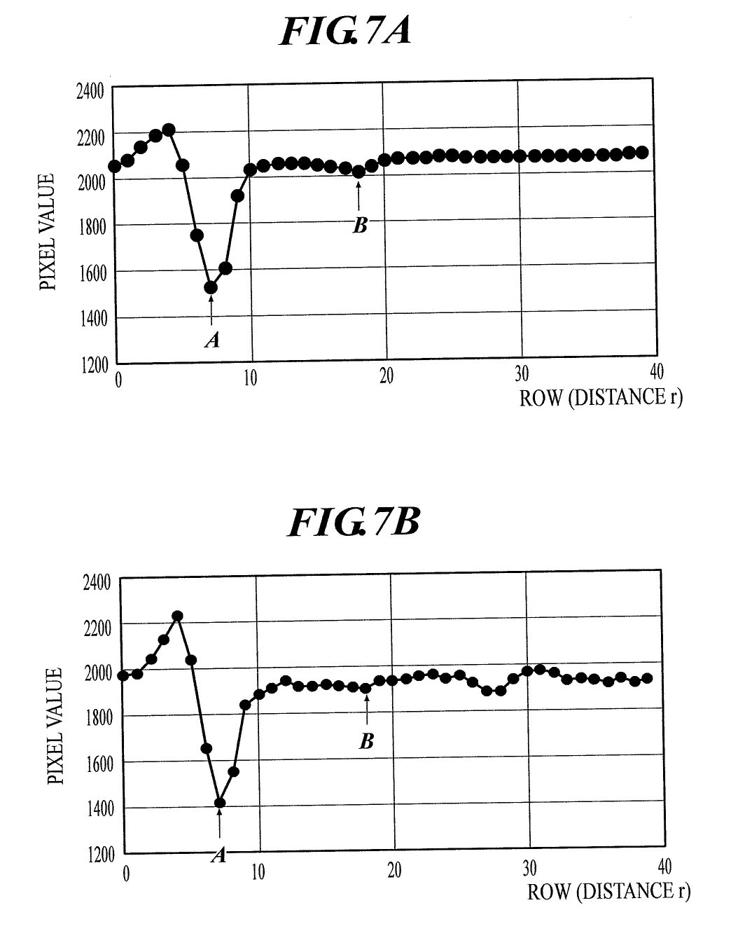

The control unit 21 may determine the position of the end of the bone or the end of the cartilage by calculating representative pixel values of predetermined ranges in each row corresponding to each distance r from the center of the arc in each column corresponding to each angle θ around the center of the arc in the polar coordinate converted image and referring to the calculated representative pixel values of predetermined ranges in each row (see FIG. 7A).

This is because the extreme values corresponding to the end of the bone or the end of the cartilage may not be obvious depending on the conditions of the joint bone and joint cartilage of the subject or patient and the brightness of the differential phase image, for example. To offset this, the control unit 21 smoothens the representative pixel values of multiple pixels (350 pixels, for example) in each predetermined range centered around a pixel in the row corresponding to each distance r from the center of the arc in each column corresponding to each angle θ around the center of the arc in the polar coordinate converted image (see FIG. 7A). The predetermined range may be defined in each row or across adjacent rows.

This clearly defines the pixel in the row corresponding to the provisional position of the end of the bone (indicated by arrow A) and having an extreme value (which is the minimum value in FIG. 7A) at the position closest to the center of the arc, and the pixel in the row corresponding to the provisional position of the end of cartilage (indicated by arrow B) and having the subsequent extreme value to that of the end of the bone (which is the minimum value in FIG. 7A). The control unit 21 then refers to the provisional positions of the end of the bone and the end of the cartilage to estimate the actual positions of the end of the bone and the end of the cartilage and determines the actual positions of the end of the bone and the end of the cartilage to be within a predetermined number of ranges centered at the provisional positions of the end of the bone and the end of the cartilage along the direction of the rows in each column corresponding to each angle θ around the arc in the polar coordinate converted image, for example.

FIG. 7A is a graph illustrating representative pixel values of the predetermined ranges in the rows corresponding to the distances r from the center of the arc in a predetermined column corresponding to a predetermined angle θ around the arc in the polar coordinate converted image. FIG. 7B is a graph illustrating the pixel values in the rows corresponding to the distances r from the center of the arc in the predetermined column in the polar coordinate converted image in FIG. 7A.

The control unit (calculator) 21 then calculates the thicknesses of the cartilage in multiple columns corresponding to multiple angles θ around the arc, on the basis of the positions of the end of the bone and the end of the cartilage in each column corresponding to each angle θ around the arc in the polar coordinate converted image. That is, the control unit 21 calculates the distances between the positions (rows) of the end of the bone and the positions (rows) of the end of the cartilage in multiple columns in the polar coordinate converted image, as the thicknesses of the cartilage. The control unit 21 then calculates the standard deviation (degree of variance) of the calculated thicknesses of the cartilage in the columns in the polar coordinate converted image as a feature value relevant to the condition of the soft tissue along the direction of the angle θ around the arc.

The degree of variance in the thicknesses of the cartilage may be determined through dispersion, for example, besides the standard deviation.

Each angle θ around the center of the arc in the polar coordinate converted medical image corresponds to a straight line (normal line) substantially orthogonal to the contour of the end of the bone in the medical image. The control unit (determiner) 21 determines the positions of the end of the bone and the end of the cartilage in columns corresponding to the straight lines.

The control unit (calculator) 21 calculates the first feature value relevant to the condition of the soft tissue extending along the contour of the end of the bone in reference to the pixel values on the straight lines substantially orthogonal to the end of the bone in the medical image. In detail, the control unit 21 calculates the thicknesses of the soft tissue in each of columns on the basis of the positions of the end of the bone and the end of the soft tissue in the column in the medical image, and thereby calculates the first feature value being the degree of variance in the calculated thicknesses of the soft tissue between the columns.

The control unit 21 instructs the display unit (output unit) 23 to display the standard deviation of the thicknesses of the cartilage calculated as the feature value relevant to the condition of the soft tissue along the direction of the angle θ around the center of the arc, on the display (see FIG. 8C). At this time, the display unit 23 may also display the thicknesses of the cartilage in multiple columns corresponding to multiple angles around the center of the arc in the polar coordinate converted image.

In this way, a user can confirm the condition of the soft tissue in the entire joint along the direction of the angle θ around the center of the arc in the polar coordinate converted image, i.e., the degree of variance in the thickness of the entire cartilage.

The first scheme processes all the columns corresponding to the predetermined angles θ around the center of the arc in the polar coordinate converted image. Not all of the columns require processing if only the tendency of the variation in the thickness of the entire cartilage is to be determined, for example. High-speed processing can be achieved through calculation of the thickness of the cartilage by assigning columns at a predetermined pitch (every other column) in the polar coordinate converted image, for example.

<Second Scheme>

In the second scheme, the control unit 21 calculates a feature value relevant to the condition of the soft tissue along the direction of a distance r from the center of the arc in the polar coordinate converted image. In detail, the control unit 21 selects a reference column corresponding to one angle θ around the center of the arc in the polar coordinate converted image and calculates the difference between the pixel value on a row corresponding to a predetermined distance r from the center of the arc in the column and the representative pixel value of multiple rows in a predetermined number of columns surrounding (lining up) the reference column (with the reference column being a reference). This difference is calculated for a plurality of rows corresponding to predetermined distances r from the center of the arc in the column, as a feature value (second feature value) relevant to the condition of the soft tissue at the distance r from the center of the arc.

The second feature value is relevant to the condition of the soft tissue along the straight line (normal line) extending substantially orthogonal to the end of the bone (which will be described in detail below).

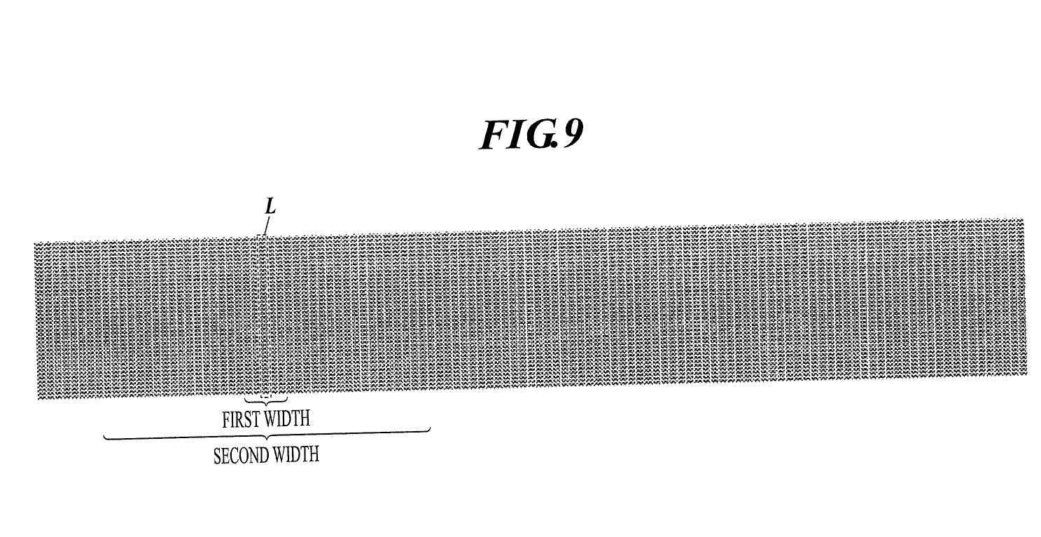

Similar to the first scheme, the control unit (retriever) 21 retrieves the differential phase image (see FIG. 3B) reconstructed by the controller 19 of the X-ray Talbot image capturing apparatus 1 via the communication unit 24. The control unit (determiner) 21 determines the position of the end of the bone in the retrieved differential phase image. The control unit (polar coordinate converter) 21 approximates the end of the bone determined in the differential phase image to an arc and converts the coordinates of the arc to polar coordinates consisting of a component of the angle θ around the center of the arc and a component of the distance r from the center of the arc (see FIG. 9).

FIG. 9 illustrates the portion of the polar coordinate converted image illustrated in FIG. 5A converted to pixel values in accordance with a predetermined gradation (such as a 16-bit gradation), as in FIG. 5B.

Through the second scheme like the first scheme, the control unit 21 needs not to use the pixel values of the entire polar coordinate converted image for the direction of the r-axis. Thus, the control unit 21 retrieves pixel values of the pixels within a predetermined range at least from near the end of the bone toward the cartilage (in the direction away from the center of the arc or toward the bottom of FIG. 4). The scheme of determining the position of the end of the bone is the same as that in the first scheme, and thus description thereof will be omitted.

The control unit (calculator) 21 selects a reference column corresponding to at least one angle θ around the center of the arc in the polar coordinate converted image and calculates the difference between the pixel value of a predetermined row corresponding to a predetermined distance r from the center of the arc in the column and a representative pixel value of rows in a predetermined number of columns surrounding (lining up) the reference column (with the reference column as a positional reference). This difference is calculated for a plurality of rows corresponding to predetermined distances r from the center of the arc in the column, as a feature value relevant to the condition of the soft tissue along the direction of the distance r from the center of the arc.

In specific, the control unit 21 can calculate the feature value relevant to the condition of the soft tissue along the direction of the distance r from the center of the arc for only one of the columns corresponding to a predetermined angle θ around the center of the arc in the polar coordinate converted image or for each of the multiple columns corresponding to multiple angles θ around the center of the arc in the polar coordinate converted image.

In a first case in which a user is to observe the condition of a portion of the soft tissue in the joint, the feature value for the portion is calculated, the feature value being relevant to the condition of the soft tissue in the portion along the direction of the distance r from the center of the arc. In a second case in which a user is to observe the condition of the entire soft tissue in the joint, the feature value is calculated for multiple columns in the polar coordinate converted image, the feature value being relevant to the condition of the soft tissue along the direction of the distance r from the center of the arc.

<First Case>

In detail, a user assigns an area in the joint (the end of the bone, for example) in which the condition of the soft tissue is to be observed in the differential phase image or the polar coordinate converted image, for example. In response, the control unit 21 determines a central column (column L surrounded by the dashed line in FIG. 9) of the multiple columns in the area assigned by the user in the polar coordinate converted image. The control unit 21 then calculates representative pixel values in a row remote from the center of the arc by a distance r, in first and second groups each consisting of a different number of columns surrounding (lining up) the central column L. The control unit 21 then calculates the difference between the representative pixel values in the row in the first and second groups.

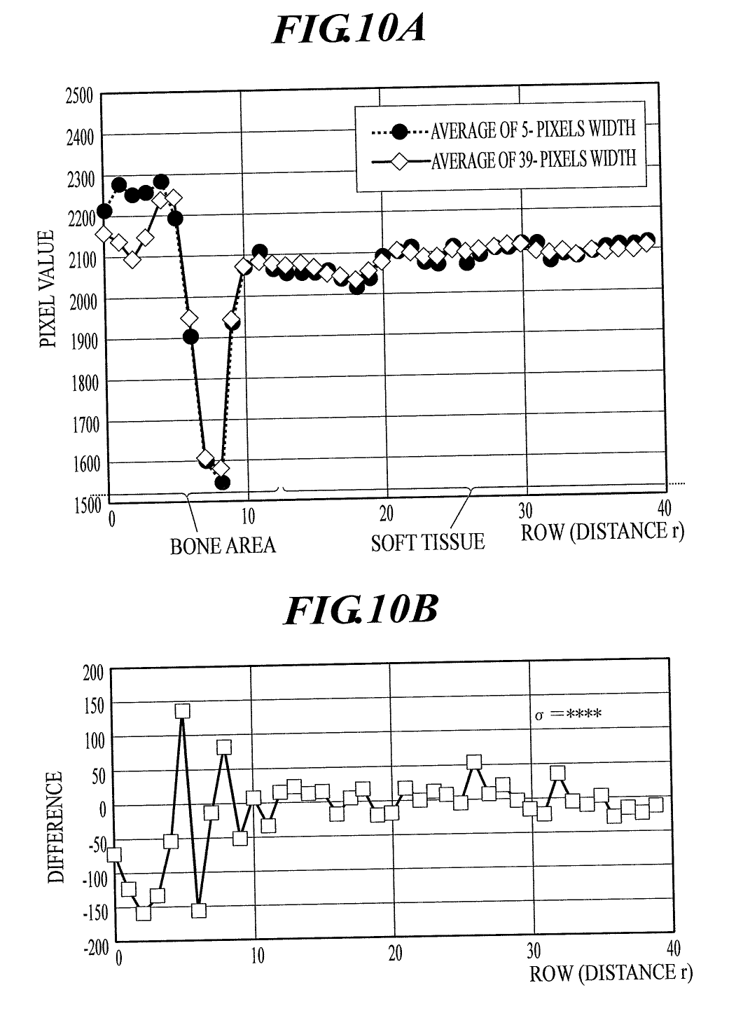

The control unit 21 defines a first range having a 5-pixels width (in the horizontal direction in FIG. 9) with the determined column as a center and a second range having a 39-pixels width with the determined column as a center in each row corresponding to a distance r from the center of the arc. The control unit 21 then calculates the averages of the pixel values in the first and second ranges as representative values (see FIG. 10A). The control unit 21 then calculates the difference between the average pixel value of the first range and the average pixel value of the second range in each row corresponding to a distance r from the center of the arc. In this way, the second range, which includes a relatively large number of pixels, provides the overall tendency without the influence of local noise and error. The portions corresponding to the end of the bone and the end of the cartilage is indistinctive through calculation of the difference between the representative pixel values of the first and second ranges having different widths.

The first range may have any width smaller than that of the second range besides a 5-pixel width. Alternatively, the first range may have a width of one pixel. The number of pixels in the first and second ranges may be modified appropriately to any other number.

The control unit 21 instructs the display unit (output unit) 23 to display the difference among multiple rows corresponding to respective distances r from the center of the arc in the polar coordinate converted image as feature values relevant to the condition of the soft tissue along the direction of the distances r from the arc in the determined column, on a display.

FIG. 10A is a graph illustrating the average pixel values of the first and second ranges in each row corresponding to a distance r from the center of the arc. FIG. 10B is a graph illustrating the difference between the average pixel values of the first and second ranges in each row corresponding to a distance r from the center of the arc.

Alternatively, the control unit 21 may calculate the standard deviation (degree of variance) of the differences among multiple rows corresponding to multiple distances r from the center of the arc in the polar coordinate converted image as a feature value relevant to the condition of the soft tissue along the direction of the distance r from the center of the arc.

Alternatively, the control unit 21 may calculate the standard deviation of the differences among the area corresponding to the soft tissue in the determined column in the polar coordinate converted image, as a feature value relevant to the condition of the soft tissue along the direction of the distance r from the center of the arc. In detail, the control unit 21 determines the position of at least one of the end of the bone and the end of the cartilage in the determined column in the polar coordinate converted image, as in the first scheme. The control unit 21 then determines the area corresponding to the soft tissue with reference to at least one of the positions of the end of the bone and the end of the cartilage determined in the multiple rows corresponding to the multiple distances r from the center of the arc in the determined column in the polar coordinate converted image. If the reference is the position of the end of the bone, the control unit 21 determines a position (row) shifted from the row corresponding to the position of the end of the bone among the multiple rows corresponding to the multiple distances r from the center of the arc, by a predetermined number of rows toward the cartilage (away from the center of the arc), as the boundary between the area corresponding as the bone and the area corresponding to the soft tissue. If the reference is the position of the end of the cartilage, the control unit 21 determines a position (row) shifted from the row corresponding to the position of the end of the cartilage among the multiple rows corresponding to the multiple distances r from the center of the arc, by a predetermined number of rows toward the bone (toward the center of the arc), as the boundary between the area corresponding to the bone and the area corresponding to the soft tissue.

The control unit 21 then calculates the standard deviation of the differences in only the multiple rows in the area corresponding to the soft tissue among the multiple rows corresponding to the distances r from the center of the arc in the determined column in the polar coordinate converted image.

The degree of variance may be determined through dispersion, for example, besides the standard deviation, as in the first scheme.

The control unit 21 may also instruct the display unit 23 to display the standard deviation of the differences of the multiple rows corresponding to the multiple distances r from the center of the arc in the polar coordinate converted image or the standard deviation of the differences of only the multiple rows in the area corresponding to the soft tissue in the polar coordinate converted image, on the display (see FIG. 10B).

This allows a user to confirm the condition of the soft tissue in a portion of the joint along the direction of the distance r from the center of the arc, i.e., the absence of asperity on the surface of a portion of the cartilage in the joint and the distinctness of the position of the end of the cartilage, in the polar coordinate converted image.

<Second Case>

In detail, the control unit 21 calculates the representative pixel values for first and second groups of columns each consisting of a different number of columns surrounding (lining up) the respective columns corresponding to different angles θ around the center of the arc in the polar coordinate converted image, in each row corresponding to a distance r from the center of the arc in the determined column. The control unit 21 then calculates the differences between the representative pixel values of the first and second groups of columns in each row. In specific, the control unit 21 repeats the same process carried out in the first case to a column corresponding to an angle θ around the center of the arc in the polar coordinate converted image, on the columns corresponding to the angles θ around the center of the arc in the polar coordinate converted image, after shift of the target column by one pixel at a time in the direction of the angle θ, for example.

The control unit 21 then calculates the standard deviation (degree of variance) of the calculated differences in the multiple rows corresponding to multiple distances r from the center of the arc in the columns corresponding to the angles θ around the center of the arc in the polar coordinate converted image, as a feature value relevant to the condition of the soft tissue along the direction of the distance r from the center of the arc.

The control unit 21 may calculate the standard deviation of the differences in only the area corresponding to the soft tissue in multiple columns in the polar coordinate converted image, as a feature value relevant to the condition of the soft tissue along the direction of the distance r from the center of the arc, as in the first case.

Since the distances r from the center of the arc in the polar coordinate converted medical image correspond to the positions on the straight lines (normal lines) substantially orthogonal to the end of the bone in the medical image, as described above, the control unit (calculator) 21 calculates a second feature value relevant to the condition of the soft tissue along the normal lines using the pixel values on the straight lines (normal lines) substantially orthogonal to the end of the bone in the medical image. In detail, the control unit 21 selects a reference column corresponding to one of the straight lines substantially orthogonal to the end of the bone in the medical image and calculates the second feature values being the difference between the reference pixel value of the reference column and the pixel value of each of a predetermined number of columns surrounding (lining up) the reference column, in each of rows defined by different positions on the straight lines.

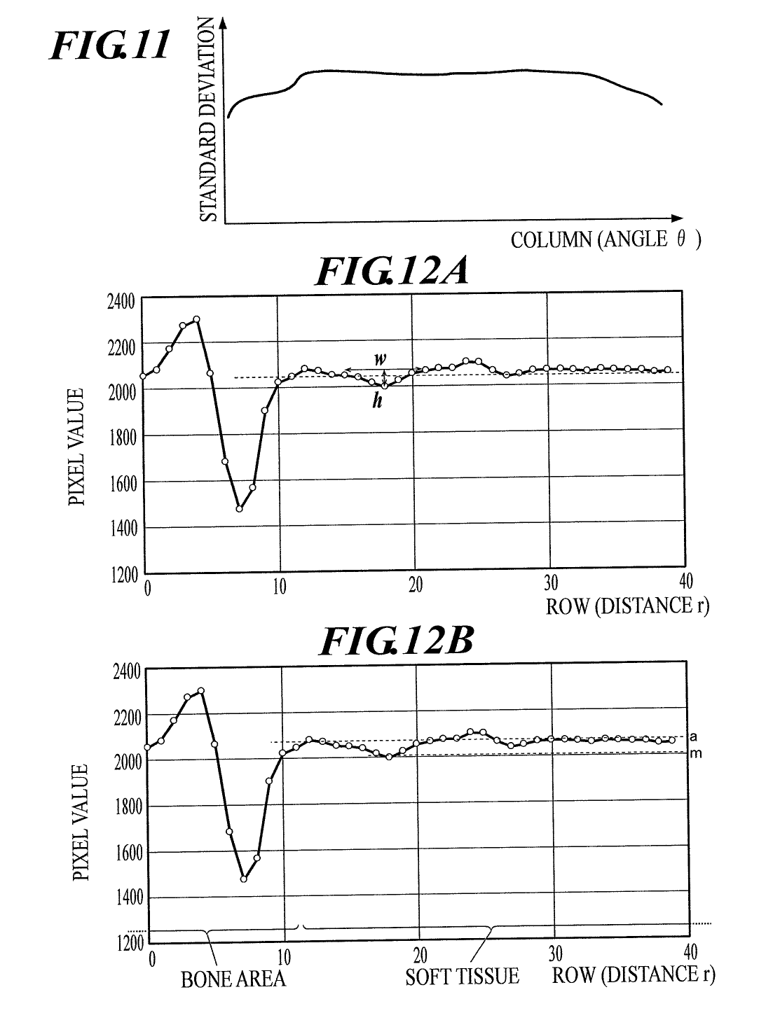

The control unit 21 may instruct the display unit 23 to display the calculated standard deviations with respect to the multiple columns in the polar coordinate converted image, on the display (see FIG. 11). FIG. 11 is a graph illustrating the standard deviations of the multiple columns in the polar coordinate converted image.

This allows a user to confirm the condition of the entire soft tissue in the joint along the direction of the distance r from the center of the arc, i.e., the absence of asperity on the surface of the entire cartilage in the joint and the distinctness of the position of the end of the cartilage, in the polar coordinate converted image.

The degree of variance may be determined through dispersion, for example, besides the standard deviation, as in the first scheme.

The second scheme processes all the columns corresponding to the predetermined angles θ around the center of the arc in the polar coordinate converted image. Not all of the columns require processing if only the tendency of the asperity on the surface of the entire cartilage is to be determined, for example. High-speed processing can be achieved through calculation of the degree of variance by assigning columns at a predetermined pitch (every other column) in the polar coordinate converted image, for example.

The control unit 21 may switch between the first and second cases in response to a user operation of the operating unit 22. The display unit 23 switches the feature values relevant to the condition of the soft tissue along the direction of the distance r from the center of the arc calculated in the first and second cases, to appear on the display.

<Third Scheme>

The third scheme also calculates a feature value (second feature value) relevant to the condition of the soft tissue along the direction of the distance r from the center of the arc in the polar coordinate converted image like the second scheme, using pixel values in only one of the columns corresponding to an angle θ around the center of the arc in the polar coordinate converted image (described below), unlike the second scheme. In detail, the control unit 21 selects a reference column corresponding to at least one angle θ around the center of the arc in the polar coordinate converted image and calculates a feature value relevant to the condition of the soft tissue in the reference column along the direction of the distance r through a predetermined calculation process based on pixel values of multiple rows surrounding (lining up) a row representing the position of the end of the cartilage and corresponding to a distance r from the center of the arc.

In specific, like the first and second schemes, the control unit (retriever) 21 retrieves the differential phase image (see FIG. 3B) reconstructed by the controller 19 of the X-ray Talbot image capturing apparatus 1 via the communication unit 24. The control unit (determiner) 21 determines the position of the end of the bone on the basis of the retrieved differential phase image. The control unit (polar coordinate converter) 21 approximates the end of the bone determined in the differential phase image to an arc and converts the coordinates of the arc to polar coordinates consisting of a component of the angle θ around the center of the arc and a component of the distance r from the center of the arc (see FIG. 9).

Through the third scheme like the first and second schemes, the control unit 21 needs not to use the pixel values of the entire polar coordinate converted image for the direction of the r-axis. Thus, the control unit 21 retrieves the pixel values of the pixels within a predetermined range at least from near the end of the bone toward the cartilage (in the direction away from the center of the arc or toward the bottom of FIG. 4).

The control unit (determiner) 21 selects a reference column corresponding to at least one angle θ around the center of the arc in the polar coordinate converted image and determines a row corresponding the distance r from the center of the arc representing the position of the end of the cartilage in the reference column, on the basis of the pixel values in the polar coordinate converted image (see FIG. 5B), as in the first and second schemes.

In specific, like the first case in the second scheme, a user selects an area in the joint (the end of the bone, for example) in which the condition of the soft tissue is to be observed in the differential phase image or the polar coordinate converted image, for example. In response, the control unit 21 determines one of the columns (column L surrounded by the dashed line in FIG. 9) in the area selected by the user in the polar coordinate converted image. The control unit 21 then determines the row representing the position of the end of the cartilage among the multiple rows corresponding to the direction of the distance r from the center of the arc in the column determined in the polar coordinate converted image.

The third scheme manually or automatically calculates the feature value relevant to the condition of the soft tissue along the direction of the distance r from the center of the arc in the polar coordinate converted image. The manual and automatic calculation processes of the feature value will now be described in detail below.

<Manual Calculation>

In the manual calculation of the feature value, the control unit 21 instructs the display unit 23 to display a graph illustrating the pixel values of the rows corresponding to the distances r from the center of the arc in the column determined in the polar coordinate converted image, on a display (see FIG. 12A). A user operates the operating unit 22 to select multiple rows surrounding (lining up) the row determined to represent the position of the end of the cartilage, on the graph. The selected multiple rows include at least the row representing the position of the end of the cartilage, i.e., the extreme value (minimum value in FIG. 12A) corresponding to the position of the end of the cartilage and adjacent rows having pixel values smaller than those of the surrounding areas, for example. The operating unit 22 then generates an operational signal corresponding to the user operation and outputs the operational signal to the control unit 21.

The operating unit 22 and the control unit 21 constitute a first assigner that assigns multiple rows surrounding (lining up) the row representing the position of the end of the cartilage in the column in the polar coordinate converted image, in response to a user operation.

The control unit (calculator) 21 then calculates the difference h between the pixel value of the row representing the position of the end of the cartilage and the representative pixel value of the assigned rows in the column corresponding to an angle θ in the polar coordinate converted image and calculates the ratio h/w of the difference h to the number of selected rows w as the feature value relevant to the condition of the soft tissue along the direction of the distance r from the center of the arc.

In detail, the control unit 21 calculates the average of the pixel values of the assigned rows in response to a user operation of the operating unit 22 and calculates the difference h between the average and the pixel value of the row representing the position of the end of the cartilage, for example. The difference h may be an absolute value if the pixel value of the position of the end of the cartilage is a maximum value.

The control unit 21 calculates the number of rows w selected in response to a user operation of the operating unit 22. The control unit 21 then calculates the ratio h/w of the difference h to the number of rows w as a feature value relevant to the condition of the soft tissue along the direction of the distance r from the center of the arc and instructs the display unit (output unit) 23 to display the feature value on the display.

The ratio h/w of the difference h to the number of rows w is large if the position of the end of the cartilage is clear, whereas the ratio h/w is small if the surface of the cartilage is asperous and the position of the end of the cartilage is unclear.

<Automatic Calculation>

In the automatic calculation of the feature value, the control unit (second assigner) 21 automatically assigns multiple rows corresponding to the soft tissue adjacent to a row representing the position of the determined end of the cartilage in the column corresponding to an angle θ in the polar coordinate converted image. In specific, the control unit 21 determines the multiple rows in the area corresponding to the soft tissue among the multiple rows corresponding to the distances r from the center of the arc in the column in the polar coordinate converted image, on the basis of the position of the end of the cartilage, as in the second scheme.

The control unit 21 then calculates the absolute difference of the pixel value m of the row representing the position of the end of the cartilage and the representative pixel value of the assigned rows, in the column in the polar coordinate converted image, as the feature value relevant to the condition of the soft tissue along the direction of the distance r from the center of the arc. In detail, after calculating the average a of the pixel values of the assigned rows, the control unit 21 calculates the absolute difference |m−a| of the pixel value m of the row representing the position of the end of the cartilage and the average a of the pixel values of the multiple rows as a feature value relevant to the condition of the soft tissue along the direction of the distance r from the center of the arc, for example. The control unit 21 then instructs the display unit (output unit) 23 to display the feature value on the display, for example. The difference is not always an absolute value if the pixel value at the position of the end of the cartilage is a maximum value.

The resulting absolute difference |m−a| is large if the position of the end of the cartilage is clear, whereas the absolute difference |m−a| is small if the surface of the cartilage is asperous and the position of the end of the cartilage is unclear.

The control unit (determiner) 21 selects a column corresponding to one of the straight lines substantially orthogonal to the end of the bone in the medical image and determines a row corresponding to one of the straight lines and representing the position of the end of the soft tissue.

The control unit (calculator) 21 calculates the second feature value through a predetermined calculation process based on the pixel values of multiple rows surrounding (lining up) the determined row representing the position of the end of the soft tissue in the reference column in the medical image.

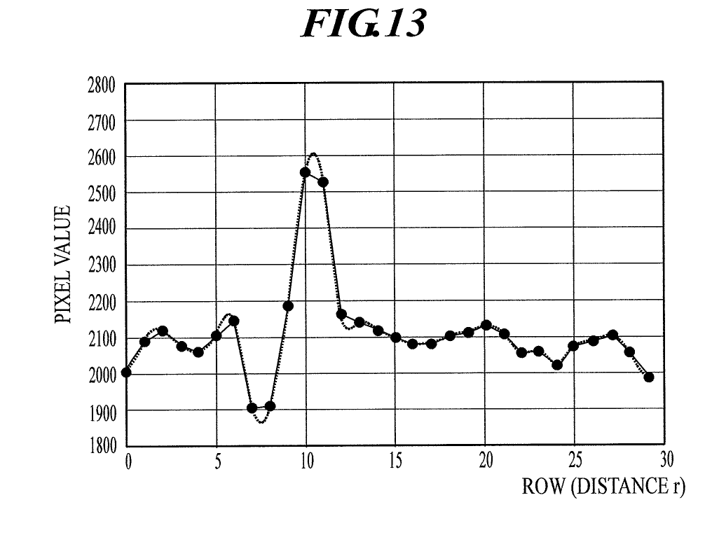

The control unit (interpolator) 21 may select a reference column corresponding to an angle θ around the center of the arc in the polar coordinate converted image and interpolate the pixel values between multiple rows corresponding to the distances r from the center of the arc in the reference column through a predetermined technique (such as spline interpolation) (see FIG. 13).

The control unit 21 may determine an area corresponding to the soft tissue on the basis of the position of at least one of the end of the bone and the end of the cartilage in multiple rows corresponding to multiple distances r from the center of the arc in the reference column in the polar coordinate converted image and perform spline interpolation by reference to only the pixel values of the rows in the area corresponding to the soft tissue. The area corresponding to the soft tissue is determined through the same scheme as the second scheme, and thus detailed descriptions are omitted.

The control unit 21 then may calculate the feature value relevant to the condition of the soft tissue along the direction of the distance r from the center of the arc in the column in which the pixel values between rows have been interpolated in the polar coordinate converted image, through the predetermined calculation process.

FIG. 13 is a graph illustrating the pixel values in each row corresponding to each distance r from the center of the arc in the reference column in the polar coordinate converted image, where the solid line represents the pixel values before interpolation and the dotted line represents the pixel values after interpolation.

Spline interpolation exemplifies a technique of interpolation of the pixel values. Alternatively, any other technique of interpolation may be used.

The control unit 21 may carry out a process, which is the same as that carried out on the reference column corresponding to an angle θ around the center of the arc in the polar coordinate converted image, on the multiple columns corresponding to multiple angles θ around the center of the arc in the polar coordinate converted image, to calculate feature values relevant to the condition of the soft tissue along the direction of the distance r from the center of the arc.

As described above, the medical image processing apparatus 2 of the medical image capturing system 100 according to this embodiment appropriately calculates feature values relevant to the condition of soft tissue in an absorption image, a differential phase image, a small-angle scattering image and the like reconstructed from a medical image or a moire image of the bone and soft tissue of a subject captured by the X-ray Talbot image capturing apparatus 1, and quantitatively measures the condition of the soft tissue. In specific, the medical image processing apparatus 2 refers to pixel values on a straight line (normal line) substantially orthogonal to the end of the bone in the medical image to calculate a first feature value relevant to the condition of the soft tissue along a direction corresponding to the direction extending along the contour of the end of the bone and a second feature value relevant to the condition of the soft tissue along the extending direction of the straight line substantially orthogonal to the end of the bone.

In detail, the medical image processing apparatus 2 can refer to the pixel values in a polar coordinate converted medical image (polar coordinate converted image) to calculate a feature value (first feature value) relevant to the condition of the soft tissue along the direction of the angle θ around the center of the arc in the polar coordinate converted image and quantitatively measure the condition of the soft tissue along the direction of the angle θ around the center of the arc in the polar coordinate converted image. With reference to the pixel values in the polar coordinate converted image, the medical image processing apparatus 2 calculates a feature value (second feature value) relevant to the condition of the soft tissue along the direction of the distance r from the center of the arc in the polar coordinate converted image and quantitatively measures the condition of the soft tissue along the direction of the distance r from the center of the arc in the polar coordinate converted image. In this way, depressions and/or asperity on the surface of the cartilage can be appropriately observed if the position of the end of the joint cartilage is unclear, for example.

The first scheme can appropriately determine the positions of the end of the bone and the end of the cartilage in each column corresponding to the angles θ around the center of the arc in the polar coordinate converted image, on the basis of the pixel values in the polar coordinate converted image. The thicknesses of the cartilage in multiple columns corresponding to multiple angles θ around the center of the arc in the polar coordinate converted image are calculated. The degree of variance of the calculated thicknesses of the cartilage in the columns is calculated as a feature value (first feature value) relevant to the condition of the soft tissue along the direction of the angle θ around the center of the arc in the polar coordinate converted image. In this way, a user can observe the condition of the soft tissue in the entire joint along the direction of the angle θ around the center of the arc in the polar coordinate converted image, i.e., the degree of variance in the thickness of the entire cartilage.

The second scheme calculate a feature value (second feature value) relevant to the condition of the soft tissue along the direction of the distance r from the center of the arc in the polar coordinate converted image in a reference column corresponding to at least one angle θ around the center of the arc in the polar coordinate converted image.

In detail, the representative pixel values of the first and second ranges of a reference column in each row corresponding to each respective distance r from the center of the arc in the polar coordinate converted image are calculated. The differences between the representative pixel values of the first range and the respective representative pixel values of the second range in the rows are calculated as feature values relevant to the condition along the direction of the distance r from the center of the arc in the column. This allows a user to confirm the condition of a portion of the soft tissue in the joint along the direction of the distance r from the center of the arc, i.e., the absence of asperity on the surface of a portion of the cartilage and the distinctness of the end of the cartilage, in the polar coordinate converted image. The degree of variance in the differences in the multiple rows calculated as a feature value enables simple confirmation of the condition of the soft tissue along the direction of the distance r from the center of the arc in the polar coordinate converted image, in a portion of the joint.

The differences for the multiple rows calculated as above for the multiple columns corresponding to multiple angles θ around the center of the arc in the polar coordinate converted image are calculated as feature values relevant to the condition along the direction of the distance r from the center of the arc. This allows a user to confirm the condition of the soft tissue in the entire joint along the direction of the distance r from the center of the arc, i.e., the absence of asperity on the surface of the entire cartilage and the distinctness of the end of the cartilage, in the polar coordinate converted image. The degree of variance in the differences in the multiple rows in the multiple columns calculated as a feature value enables simple confirmation of the condition of the soft tissue in the entire joint along the direction of the distance r from the center of the arc in the polar coordinate converted image.

The third scheme calculates a feature value (second feature value) relevant to the condition of the soft tissue along the direction of the distance r from the center of the arc through a predetermined calculation process based on the pixel values of multiple rows surrounding (lining up) the row representing the position of the end of the cartilage in a reference column corresponding to at least one angle θ around the center of the arc in the polar coordinate converted image.

In detail, the ratio of the difference between the pixel value of the row representing the position of the end of the cartilage and the representative pixel value of the rows selected by a user operation to the number of rows is calculated for the reference column of the polar coordinate converted image, as a feature value relevant to the condition of the soft tissue along the direction of the distance r from the center of the arc. Alternatively, the difference between the pixel value of the row representing the position of the end of the cartilage and the representative pixel value of the automatically assigned rows is calculated for the reference column in the polar coordinate converted image, as a feature value relevant to the condition of the soft tissue along the direction of the distance r from the center of the arc. This allows a user to confirm the condition of a portion of the soft tissue in a portion of the joint along the direction of the distance r from the center of the arc, i.e., the absence of asperity on the surface of a portion of the cartilage and the distinctness of the end of the cartilage, in the polar coordinate converted image.

Interpolation of the pixel values between rows corresponding to distances r from the center of the arc in the reference column in the polar coordinate converted image achieves appropriate calculation of a feature value relevant to the condition of the soft tissue along the direction of the distance r from the center of the arc.

Determination of an area corresponding to the soft tissue in the reference column in the polar coordinate converted image achieves appropriate calculation of a feature value relevant to the condition of the soft tissue along the direction of the distance r from the center of the arc, such as the degree of variance.

The representative pixel value of the predetermined range in each row corresponding to a distance r from the center of the arc in a reference column corresponding to at least one angle θ around the center of the arc in the polar coordinate converted image is calculated and the representative pixel value of the predetermined range in each row is referred to achieve appropriate determination of the position of end of the bone and the end of the cartilage even if the extreme values corresponding to the end of the bone and the end of the cartilage are unclear depending on the condition of the bone and the cartilage in the joint of the subject or patient and/or the brightness of the differential phase image and other images, for example.

In the embodiments described above, straight lines (normal lines) substantially orthogonal to the contour of the end of the bone in the medical image correspond to the angles around the arc in the polar coordinate converted medical image. The embodiments are not limited thereto. In specific, the control unit 21 needs not to function as a polar coordinate converter that performs polar conversion of a medical image and may function as a normal-line definer that defines a normal line of the contour of the end of the bone in the medical image.

The control unit 21 of the medical image processing apparatus 2 according to the embodiments described above functions as a retriever, a determiner, a calculator, a polar-coordinate converter, a first assigner, a second assigner, and an interpolator. The control unit 21 may have any other functions. For example, the controller 19 of the X-ray Talbot image capturing apparatus 1 may include a calculating device having the functions, such as a retriever, a determiner, a calculator, a polar-coordinate converter, a first assigner, a second assigner, and an interpolator, equivalent to those of the control unit 21.

The medical image according to the embodiments described above is exemplified by an absorption image, a differential phase image, or a small-angle scattering image reconstructed from a moire image captured by the X-ray Talbot image capturing apparatus 1. Alternatively, the medical image may be any other image. In specific, the medical image should be an image of bone and soft tissue of a subject and any image captured by an image capturing apparatus employing magnetic resonance imaging (MRI) or computed tomography (CT).

The embodiment described above should not be construed to limited the present invention and may be appropriately modified without departing from the scope of the present invention.

The entire disclosure of Japanese Patent Application No. 2016-124036 is incorporated to the present application.

1. A medical image processing apparatus comprising:
a retriever that retrieves a medical image of a bone and a soft tissue of a subject;
a determiner that determines a position of at least one of an end of the bone and an end of the soft tissue based on the medical image retrieved by the retriever; and
a calculator that calculates a feature value relevant to a condition of the soft tissue based on the position of at least one of the end of the bone and the end of the soft tissue determined by the determiner.

2. The medical image processing apparatus according to claim 1, further comprising:
a normal-line definer that defines a straight line substantially orthogonal to the end of the bone based on the medical image,
wherein the calculator calculates a first feature value relevant to a condition of the soft tissue extending along a contour of the end of the bone by reference to pixel values of the medical image on the straight line defined by the definer.

3. The medical image processing apparatus according to claim 2, wherein,
the determiner determines the position of the end of the bone and the position of the end of the soft tissue in columns corresponding to the straight lines substantially orthogonal to a contour of the end of bone in the medical image, and
the calculator calculates a thickness of the soft tissue in each of columns based on the position of the end of the bone and the position of the end of the soft tissue in each column in the medical image and calculates the first feature value being a degree of variance in the calculated thickness of the soft tissue between the columns.

4. The medical image processing apparatus according to claim 2, further comprising:
a polar-coordinate converter that converts the medical image to polar coordinates including a component of an angle around a center of an arc being approximation of the end of the bone and a component of a distance from the center of the arc,
wherein the calculator calculates the first feature value being a feature value relevant to the condition of the soft tissue along the direction of the distance from the center of the arc by reference to the pixel values of the medical image converted to the polar coordinates by the polar-coordinate converter.

5. The medical image processing apparatus according to claim 1 further comprising:
a normal-line definer that defines a straight line substantially orthogonal to the end of the bone based on the medical image,
wherein the calculator calculates a second feature value relevant to a condition of the soft tissue along the extending direction of the straight line by reference to pixel values of the medical image on the straight line defined by the normal-line definer.

6. The medical image processing apparatus according to claim 5, wherein the calculator selects a reference column corresponding to one of the straight lines substantially orthogonal to the end of the bone in the medical image, and calculates the second feature value being the difference between the reference pixel value of the reference column and a representative pixel value of the pixel values of a predetermined number of columns having a predetermined width with the reference column being a positional reference, in each of rows defined by different positions on the straight lines.

7. The medical image processing apparatus according to claim 6, wherein the calculator calculates a representative pixel value for a first group of columns and a representative pixel value for a second group of columns, each group of columns consisting of a different number of columns with the reference column being a positional reference, in each of rows defined by different positions on the straight lines, calculates the second feature value of the reference column, the second feature value being the difference between each representative pixel value for the first group of columns and the corresponding representative pixel value for the second group of columns in each row.

8. The medical image processing apparatus according to claim 6, wherein the calculator further calculates the second feature value being the degree of variance in the calculated differences in the rows.

9. The medical image processing apparatus according to claim 6, wherein the calculator selects multiple columns corresponding to multiple straight lines substantially orthogonal to the end of the bone in the medical image, calculates the second feature value being the difference between each pixel value of each reference column of the reference columns and a representative pixel value of the pixel values of a predetermined number of columns with the reference column being a positional reference, in each of rows defined by different positions on the straight lines.

10. The medical image processing apparatus according to claim 9, wherein the calculator further calculates the second feature value being the degree of variance in the calculated differences in the rows for each of the columns.

11. The medical image processing apparatus according to claim 5, wherein,
the determiner selects a column corresponding to one of the straight lines substantially orthogonal to the end of the bone in the medical image and determines a reference row defined by a position on the straight line and representing the position of the end of the soft tissue, and
the calculator calculates the second feature value through a predetermined calculation process based on a pixel values of multiple rows with the reference row being a positional reference which is determined by the determiner and which represents the position of the end of the soft tissue in the reference column in the medical image.

12. The medical image processing apparatus according to claim 11, further comprising:
a first assigner that assigns multiple rows with the reference row being a positional reference determined by the determiner and representing the position of the end of the soft tissue in the reference column in the medical image, in response to a user operation,
wherein the calculator calculates a difference between a reference pixel value of the reference row determined by the determiner and representing the position of the end of the soft tissue and the representative pixel value of the rows assigned by the first assigner, in the reference column in the medical image, and calculates the ratio of the calculated difference to the number of rows, as the second feature value.

13. The medical image processing apparatus according to claim 11, further comprising:
a second assigner that assigns multiple rows corresponding to the soft tissue with the reference row being a positional reference determined by the determiner and representing the position of the end of the soft tissue in the reference column in the medical image,
wherein the calculator calculates the second feature value being the difference between a reference pixel value of the reference row determined by the determiner and representing the position of the end of the soft tissue and the representative pixel value of the rows assigned by the second assigner, in the reference column in the medical image.

14. The medical image processing apparatus according to claim 11, further comprising:
an interpolator that interpolates pixel values between multiple rows corresponding to different positions on the straight line in the reference column in the medical image,
wherein the calculator carries out a predetermined calculation process on the reference column in the medical image to calculate the second feature value, the pixel values of the reference column being interpolated by the interpolator.

15. The medical image processing apparatus according to claim 5, wherein the calculator selects a reference column corresponding to one of straight lines substantially orthogonal to the end of the bone in the medical image, determines an area corresponding to the soft tissue based on the position of at least the end of the bone and the end of the soft tissue determined by the determiner in the reference column, and calculates the second feature value with respect to the determined area corresponding to the soft tissue.

16. The medical image processing apparatus according claim 5, further comprising:
a polar-coordinate converter that converts the medical image to polar coordinates including a component of an angle around the center of an arc being approximation of the end of the bone and a component of a distance from the center of the arc,
wherein the calculator calculates the second feature value being a feature value relevant to the condition of the soft tissue along the direction of the distance from the center of the arc by reference to the pixel values of the medical image converted to polar coordinates by the polar-coordinate converter.

17. The medical image processing apparatus according to claim 1, wherein the determiner selects a reference column corresponding to one of straight lines substantially orthogonal to the end of the bone in the medical image, calculates a representative pixel value in a predetermined range in every row corresponding to different positions on the selected straight line, and determines at least one of the end of the bone and the end of the soft tissue based on the calculated representative pixel value in the predetermined range in every row.

18. The medical image processing apparatus according to claim 1, further comprising:
an output unit that outputs the feature value relevant to the condition of the soft tissue calculated by the calculator.

19. The medical image processing apparatus according to claim 18, wherein the output unit further outputs the position of at least one of the end of the bone and the end of the soft tissue determined by the determiner.

20. A medical image capturing system comprising:
an image capturing apparatus which captures a bone and a soft tissue of a subject; and
an image processing apparatus, wherein
the image processing apparatus includes:
a retriever that retrieves a medical image of a bone and a soft tissue of a subject captured by the image capturing apparatus;
a determiner that determines at least one of an end of the bone and an end of the soft tissue based on the medical image retrieved by the retriever; and
a calculator that calculates a feature value relevant to a condition of the soft tissue based on at least one of the end of the bone and the end of the soft tissue determined by the determiner.
